(19) United States
(12) Patent Application Publication
(10) Pub. No.: US 2023/0301982 A1
(43) Pub. Date: Sep. 28, 2023

(54) N'-(2-CHLORO-6-METHYLBENZOYL)-4-METHYL-3-[2-(3-QUINOLYL)ETHYNYL]-BENZOHYDRAZIDE FOR TREATMENT OF ALZHEIMER'S DISEASE

(71) Applicant: SUN PHARMA ADVANCED RESEARCH COMPANY LIMITED, Mumbai, Maharashtra (IN)

(72) Inventors: Nitin Krishnaji DAMLE, Mumbai, Maharashtra (IN); Sanjay Nandlalji MANDHANE, Vadodara, Gujarat (IN); Durgesh Ashok Kumar SONI, Vadodara, Gujarat (IN); Shakti Kamalchand JAIN, Vadodara, Gujarat (IN); Vikram Krishna RAMANATHAN, Ahmedabad, Gujarat (IN)

(73) Assignee: SUN PHARMA ADVANCED RESEARCH COMPANY LIMITED, Mumbai, Maharashtra (IN)

(21) Appl. No.: 18/018,438

(22) PCT Filed: Jul. 30, 2021
(86) PCT No.: PCT/IB2021/056986

§ 371 Date: Jan. 27, 2023

(30) Foreign Application Priority Data
Jul. 31, 2020 (IN) 202021032951

(51) Int. Cl. A61K 31/4709; A61K 45/06; A61P 25/28

(52) U.S. Cl. CPC A61K 31/4709; A61K 45/06; A61P 25/28

(57) ABSTRACT

The present invention provides a method for preventing or treating Alzheimer’s disease and symptoms thereof comprising administering to a subject in need thereof a therapeutically effective amount of N′-(2-chloro-6-methylbenzoyl)-4-methyl-3-[2-(3-quinolyl)ethynyl]-benzohydrazide or a its pharmaceutically acceptable salts, wherein the prevention and/or treatment of said disease and symptoms thereof is achieved by inhibition of amyloid-beta (Aβ) plaque aggregation, tau hyperphosphorylation, c-Abl kinase or a combination thereof. Also disclosed is the use of a therapeutically effective amount of N′-(2-chloro-6-methylbenzoyl)-4-methyl-3-[2-(3-quinolyl)ethynyl]-benzohydrazide or its pharmaceutically acceptable salts to inhibit amyloid-beta (Aβ) plaque aggregation, tau hyperphosphorylation, c-Abl kinase or a combination thereof.

### FIELD OF THE INVENTION

The present invention provides a method for preventing or treating Alzheimer’s disease and symptoms thereof comprising administering to a subject in need thereof a therapeutically effective amount of N′-(2-chloro-6-methylbenzoyl)-4-methyl-3-[2-(3-quinolyl)ethynyl]-benzohydrazide or its pharmaceutically acceptable salts, wherein the prevention and/or treatment of said disease and symptoms thereof is achieved by inhibition of amyloid-beta (Aβ) plaque aggregation, tau hyperphosphorylation, c-Abl kinase or a combination thereof. Also disclosed is the use of a therapeutically effective amount of N′-(2-chloro-6-methylbenzoyl)-4-methyl-3-[2-(3-quinolyl)ethynyl]-benzohydrazide or its pharmaceutically acceptable salts to inhibit amyloid-beta (Aβ) plaque aggregation, tau hyperphosphorylation, c-Abl kinase or a combination thereof.

### BACKGROUND OF THE INVENTION

Alzheimer’s disease (AD) is a major neurodegenerative disorder predominantly present in an aging population. AD is the sixth leading cause of death in the United States and the fifth leading cause of death among Americans aged 65 and older. AD currently afflicts 5.3 million people in the United States alone.

The main features of AD are wide-spread Aβ amyloid plaques and neuronal tangles in the brain. It is known that there is a loss of synaptic plasticity resulting in cognitive deficits preceding plaque formation. Several other factors like astrogliosis, neuronal loss, and vascular alterations are also known to contribute to AD pathology. The Aβ peptide toxicity and its accumulation in brain has been partially linked to progression in many clinical studies of AD patients. The major biochemical changes that occur in AD patients are the aggregation of Aβ and abnormal processing, as well as a hyper phosphorylation of Tau.

Plaques and smaller accumulations of beta-amyloids called oligomers may contribute to the damage and death of neurons (neurodegeneration) by interfering with neuron-to-neuron communication at synapses. Tau tangles block the transport of nutrients and other essential molecules inside neurons. Although the complete sequence of events is unclear, beta-amyloid may begin accumulating before abnormal tau, and increasing beta-amyloid accumulation is associated with subsequent increases in tau. (See Neuron 2018; 98(4):861-4; JAMA Neurol 2019; 76(8):915-24).

Compounds preventing or reversing aggregation of Aβ and tau protein hyperphosphorylation therefore hold potential for the treatment of AD.

USFDA has approved five drugs for the treatment of Alzheimer’s – rivastigmine, galantamine, donepezil, memantine, and memantine combined with donepezil. With the exception of memantine, these drugs temporarily improve cognitive symptoms by increasing the amount of neurotransmitters in the brain. Memantine blocks certain receptors in the brain from excess stimulation that can damage nerve cells. The effectiveness of these drugs varies from person to person and is limited in duration. (See Alzheimer’s Dement. 2020; 16:391-460).

Despite many years of research, no clear therapeutic options are available for AD patients. In view of above facts, the inventors of the present invention have realized that there should be a pharmacologic treatment (medication) which can prevent or reverse aggregation of Aβ and tau protein hyperphosphorylation, and can provide therapeutic options for AD patients.

WO2012/098416 (“the ‘416 publication”) discloses tyrosine kinase inhibitors (e.g., Abl inhibitors) having the structural formula:

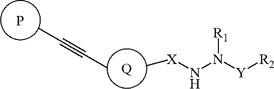

or pharmaceutically acceptable salt thereof, wherein;
ring P and ring Q are independently selected from an aryl ring having 6 to 14 carbon atoms, or a 5 to 14 membered heteroaryl ring containing one to four hetero atoms each independently selected from O, S and N, optionally substituted by one or more identical or different radicals R₃, with a proviso that when ring Q is pyridyl then ring P is heteroaryl; R₁ and R₂ are independently selected from the group consisting of hydrogen, -C₁₋₈-alkyl, —C₂—₁₀-alkenyl, -C₂₋₁₂-alkynyl, —C₃—₁₂-cycloalkyl, —C₄—₁₂-cycloalkylalkyl, —C₃—₁₂-cycloalkenyl, aryl, heteroaryl, arylalkyl and a heteroarylalkyl radical, wherein the aryl ring contains 6 to 14 carbon atoms, and the heteroaryl ring contains 5 to 14 membered ring system with one to four hetero atoms each independently selected from O, S and N, and are optionally substituted by one or more identical or different radicals R₃;
X and Y are independently selected from the group consisting of C=0, C═S and S0₂;
R₃ is selected from the group consisting of halogen, —OH, —CN, -N0₂, —N₃, -C₁₋₈-alkyl, —C₃—₁₂-cycloalkyl, -(C₁₋₈-alkyl)-C₃₋₁₂-cycloalkyl, -heterocycloalkyl containing 3 to 12 rings atoms having one or two hetero atoms each independently selected from O, S and N, —(C₁—₈-alkyl)-heterocycloalkyl containing 3 to 12 rings atoms having one or two hetero atoms each independently selected from O, S and N, -0-C₁₋₈-alkyl, -0-C₃-₁₂-cycloalkyl, —O—aryl, —O—heteroaryl, -C₁₋₈ alkyl-0-C₁₋₈alkyl, -O-C₁₋₈alkyl-0-C₁₋₈alkyl, -0-C₁₋₈alkyl-NH(C₁₋₈alkyl), -0-C_{1_8}alkyl-N(C_{1_8}alkyl)₂, -0-C_{1_8}alkyl-(heteroaryl), -C(0)-C_{1_8}alkyl, —COOH, - C(0)NH₂, -C(0)NH-C₁₋₈alkyl, -C(0)N(C₁₋₈alkyl)₂, -C(0)0-C₁₋₈alkyl, -C₁₋₈haloalkyl, -C₂₋₁₀alkenyl, -C₂₋₁₂alkynyl, —OC(0)—NH₂, -OC(0)-NH(C₁₋₈alkyl), -OC(0)-N(C₁₋₈alkyl)₂, —NH₂, -NH(C₁₋₈alkyl), -N(C₁₋₈alkyl)₂, -NH-S0₂-C₁₋₈alkyl, -N(C₁₋₈alkyl) -S0₂-C₁₋₈alkyl, — NH—C(0)-(C₁₋₈alkyl), -N(C₁₋₈alkyl)-C(0)-(C₁₋₈alkyl), —NH—C(0)0-C₁₋₈alkyl, -N(C₁₋₈alkyl)-C(0)0-C₁₋salkyl, —NH—C(0)-NH₂, —NH—C(0)-NH(C₁₋₈alkyl), -N(C₁₋₈alkyl)-C(O)- NH(C₁₋₈alkyl), -N(C₁₋₈alkyl)-C(0)-N(C₁₋₈alkyl)₂, —NH—C(0)-NH-S0₂-C₁₋₈alkyl, -N(C₁₋₈alkyl)-C(0)-NHS0₂-C₁₋₈alkyl, -N(C₁₋₈alkyl)-C(0)-N(C₁₋₈alkyl)-S0₂-C₁₋₈alkyl, -S-C₁₋₈alkyl, -S(0)-C₁₋₈alkyl, -S0₂-C₁₋₈alkyl, —S—aryl, -S(0)-aryl, S0₂-aryl, —S0₂NH₂, -S0₂NH- (C₁₋₈alkyl), -S0₂N(C₁₋₈alkyl)₂; -aryl, -(C₁₋₄alkyl)-aryl, heteroaryl or -(C₁₋₈alkyl)- heteroaryl group, wherein the aryl ring contains 6 to 14 carbon atoms, and the heteroaryl ring contains 5 to 14 membered ring system with one to four hetero atoms each independently selected from O, S and N, wherein each of the aforementioned R₃ groups may be optionally substituted with a single group selected from the group consisting of: C₁₋₄alkyl, C₁₋₄alkoxy, C₁₋₄haloalkyl, —OH, —COOH, —CN, -N0₂, halo, —NH₂ and —S0₂NH₂.

The ‘416 publication further discloses a number of specific compounds, their preparation, pharmaceutical compositions and their usefulness for the treatment of cancers such as chronic myelogenous leukemia (CML), chronic lymphocytic leukemia (CLL), acute lymphoblastic leukemia (ALL), acute myeloid leukemia (AML), myelodysplastic syndrome, melanoma, germ cell tumors, gastrointestinal stromal tumor (GIST), non-small cell lung carcinoma (SCLC), mastocytosis, neuroblastoma, glioblastoma, astrocytoma, hepatocellular carcinoma, renal cell cancer, breast cancer, cutaneous systemic sclerosis, prostate and colorectal cancer and other solid tumors.

After an exhaustive research, the inventors of the present invention have surprisingly discovered that a compound of formula:

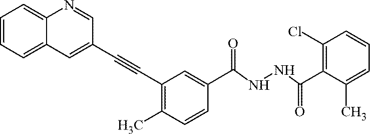 [SMILES: Cc1ccc(C(=O)NNC(=O)c2c(C)cccc2Cl)cc1C#Cc1cnc2ccccc2c1]

(referred to hereinafter as “Compound 1”), from among thousands of compounds encompassed by above Markush structure together with specifically exemplified compounds in the ‘416 publication, significantly prevents or reverse aggregation of Aβ (decreased the spread of Aβ amyloid plaques).

Hyperphosphorylated tau is a cardinal feature of AD pathology. The c-Abl activation is associated with AD tau phosphorylation both directly and through the activation of Cdk5. Hence, the use of c-Abl inhibitor could be a successful strategy for AD treatment. (Current Alzheimer Research, 2011, 8:643-651).

Thus, the present invention provides a great hope for a medication to meet the challenges of a serious global health concern, i.e., Alzheimer’s disease.

### SUMMARY OF THE INVENTION

### Problems to Be Solved by the Invention

As realized by the inventors of the present invention, there is a need for a new medication, which significantly prevents or reverses aggregation of Aβ (decreases the spread of Aβ plaques) and tau hyperphosphorylation, and at the same time possesses significantly higher brain concentration for increased efficacy and good safety profile amenable to human use.

### Means for Solving the Problems

As a result of intensive studies, the present inventors have found that Compound 1 significantly prevents and/or reverses aggregation of Aβ (decreases the spread of Aβ plaques) and may prevent tau hyperphosphorylation. In particular, Compound 1 of the present invention possesses significantly higher brain to plasma concentration and excellent safety profile amenable to human use.

Thus, in one aspect, the present invention provides:

A method for preventing or treating Alzheimer’s disease and symptoms thereof comprising administering to a subject in need thereof a therapeutically effective amount of N′-(2-chloro-6-methylbenzoyl)-4-methyl-3-[2-(3-quinolyl)ethynyl]-benzohydrazide or its pharmaceutically acceptable salts.

The method according to [1], wherein N′-(2-chloro-6-methylbenzoyl)-4-methyl-3-[2-(3-quinolyl)ethynyl]-benzohydrazide or its pharmaceutically acceptable salts regulate levels of tau hyperphosphorylation.

The method according to [1], wherein N′-(2-chloro-6-methylbenzoyl)-4-methyl-3-[2-(3-quinolyl)ethynyl]-benzohydrazide or its pharmaceutically acceptable salts regulate levels of tau hyperphosphorylation by inhibition of c-Abl-kinase.

The method according to [1], wherein N′-(2-chloro-6-methylbenzoyl)-4-methyl-3-[2-(3-quinolyl)ethynyl]-benzohydrazide or its pharmaceutically acceptable salts prevent or reverse aggregation of Aβ (decreased the spread of Aβ plaques).

The method according to [1], wherein N′-(2-chloro-6-methylbenzoyl)-4-methyl-3-[2-(3-quinolyl)ethynyl]-benzohydrazide or its pharmaceutically acceptable salts prevent or reverse aggregation of Aβ (decreased the spread of Aβ plaques) by inhibition of c-Abl-kinase.

The method according to any one of [1] to [5], wherein N(2-chloro-6-methylbenzoyl)-4-methyl-3-[2-(3-quinolyl)ethynyl]-benzohydrazide or its pharmaceutically acceptable salt is administered at a dose in the range of about 1.0 mg/kg to about 10.0 mg/kg.

A method for inhibiting tau hyperphosphorylation comprising administering to a subject an amount of compound sufficient to inhibit tau hyperphosphorylation, wherein said compound modulates an ATP-dependent enzyme, wherein said compound is N′-(2-chloro-6-methylbenzoyl)-4-methyl-3-[2-(3-quinolyl)ethynyl]-benzohydrazide or its pharmaceutically acceptable salt.

A method for inhibiting, preventing or reversing aggregation of Aβ comprising administering to an amount of compound sufficient to reduce Aβ plaque, wherein said compound modulates an ATP-dependent enzyme, wherein said compound is N′-(2-chloro-6-methylbenzoyl)-4-methyl-3-[2-(3-quinolyl)ethynyl]-benzohydrazide or its pharmaceutically acceptable salt.

The method according [7] and [8], wherein the ATP-dependent enzyme is a kinase.

The method according to [9] wherein the kinase is a tyrosine kinase.

The method according to [10] wherein the tyrosine kinase is c-Abl kinase.

The method according to any one of [1] to [11], wherein N′-(2-chloro-6-methylbenzoyl)-4-methyl-3-[2-(3-quinolyl)ethynyl]-benzohydrazide or its pharmaceutically acceptable salts is further administered in combination with an additional therapeutic agent.

The method according to [12], wherein the additional therapeutic agent according is selected from memantine, donepezil (Aricept®), galantamine (Reminyl®), tacrine hydrochloride (Cognex®), or rivastigmine tartrate (Exelon®).

N′-(2-chloro-6-methylbenzoyl)-4-methyl-3-[2-(3-quinolyl)ethynyl]-benzohydrazide or its pharmaceutically acceptable salts for use in the prevention or treatment of Alzheimer’s disease and symptoms thereof.

A pharmaceutical composition comprising a therapeutically effective amount of N′-(2-chloro-6-methylbenzoyl)-4-methyl-3-[2-(3-quinolyl)ethynyl]-benzohydrazide or its pharmaceutically acceptable salts for use in the prevention and/or treatment of Alzheimer’s disease and symptoms thereof.

The pharmaceutical composition according to [15], wherein the pharmaceutical composition has a form selected from tablets, pellets, capsules, dispersible tablets, sachets, granules or syrups.

The pharmaceutical composition according to [16], wherein the pharmaceutical composition is a capsule and is administered orally.

The pharmaceutical composition according to [16], wherein the pharmaceutical composition is a tablet and is administered orally.

The use of N′-(2-chloro-6-methylbenzoyl)-4-methyl-3-[2-(3-quinolyl)ethynyl]-benzohydrazide or its pharmaceutically acceptable salts for the manufacture of a medicament for the prevention and/or treatment of Alzheimer’s disease and symptoms thereof.

The c-Abl inhibitor for use in the prevention and/or treatment of Alzheimer’s disease and symptoms thereof, wherein the c-Abl inhibitor is N′-(2-chloro-6-methylbenzoyl)-4-methyl-3-[2-(3-quinolyl)ethynyl]-benzohydrazide or its pharmaceutically acceptable salts.

The tau hyperphosphorylation inhibitor for use in the prevention and/or treatment of Alzheimer’s disease and symptoms thereof, wherein the tau hyperphosphorylation inhibitor is N′-(2-chloro-6-methylbenzoyl)-4-methyl-3-[2-(3-quinolyl)ethynyl]-benzohydrazide or its pharmaceutically acceptable salts.

The Aβ plaque aggregation inhibitor for use in the prevention and/or treatment of Alzheimer’s disease and symptoms thereof, wherein the inhibitor is N′-(2-chloro-6-methylbenzoyl)-4-methyl-3-[2-(3-quinolyl)ethynyl]-benzohydrazide or its pharmaceutically acceptable salts.

A method of improving symptoms of Alzheimer’s disease in a subject comprising administering to the subject a therapeutically effective dose of N′-(2-chloro-6-methylbenzoyl)-4-methyl-3-[2-(3-quinolyl)ethynyl]-benzohydrazide or its pharmaceutically acceptable salts to improve the symptoms in the subject, wherein the improvement is achieved by inhibition of Aβ plaque aggregation, tau hyperphosphorylation, c-Abl kinase or combination thereof.

The method according to [23], wherein the compound, or its pharmaceutically acceptable salts is administered at a dosage in the range of from about 1.0 mg/kg to about 10.0 mg/kg.

The method according to any one of [1] to [24] in which the symptoms of AD according are difficulty remembering recent events or conversation, disorientation, mood and behavior, difficulty speaking, swallowing, walking or cognitive disorder.

The method or use according to any of [1] to [27] wherein the percentage inhibition of c-Abl kinase is 37%.

The method or use according to any of [1] to [27] wherein the percentage inhibition of c-Abl kinase is about 37%.

The method or use according to any of [1] to [27] wherein the percentage inhibition of c-Abl kinase is at least 37%.

### BRIEF DESCRIPTION OF THE DRAWINGS

Various aspects of the present invention are illustrated by reference to the following drawings.

FIG. 1: Total Aβ content in cortex of Tg APP/PS 1 Mice using ELISA.

According to the result of ELISA, Compound 1 at 30 and 45 mg/kg dose levels showed significant reduction in total Aβ content in cortex of Tg APP/PS 1 mice compared to placebo treated group.

FIG. 2: Aβ plaque load (% area fraction) in cortex of Tg APP/PS 1 mice using IHC

According to the result of IHC, Compound 1 at 30 mg/kg dose levels significantly reduced Aβ plaque area fraction compared to placebo treated group.

FIG. 3: Contextual fear conditioning (CFC): Freezing behaviour in Tg APP/PS 1 mice.

According to the result of CFC analysis, Compound 1 showed significant increase in % freezing of mice at 30 and 45 mg/kg doses as compared to placebo treated group.

FIG. 4: Aβ₄₂ and Aβ₄₂/Aβ₄₀ ratio in TgSwDI mice using ELISA

According to the result of ELISA, Compound 1 at 15, 30, and 45 mg/kg showed dose-dependent reduction in Aβ₄₂ and ratio of Aβ₄₂/Aβ₄₀. The effect on Aβ₄₂ and ratio of Aβ₄₂/Aβ₄₀ was statistically significant at doses of 30 and 45 mg/kg as compared to placebo treated group, whereas in case of nilotinib only reduction in Aβ42/40 ratio was statistically significant.

The aforementioned aspects and embodiments, and other aspects, objects, features and advantages of the present invention will be apparent from the following detailed description.

Unless otherwise indicated, the contents of each document discussed herein is incorporated by reference in its entirety.

### DETAILED DESCRIPTION OF THE INVENTION

As used herein the following definitions apply unless clearly indicated otherwise. It should be noted that the singular forms “a” “an” and “the” include plural reference unless the context clearly dictates otherwise. As used herein, the term “about”, refers to any value which lies within the range defined by a variation of up to ±10% of the value. It should be understood that unless expressly stated to the contrary, Compound 1 chemically refers to N′-(2-chloro-6-methylbenzoyl)-4-methyl-3-[2-(3-quinolyl)ethynyl]-benzohydrazide and structurally refers to:

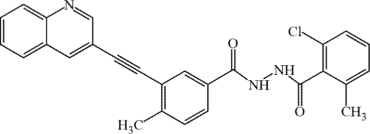 [SMILES: Cc1ccc(C(=O)NNC(=O)c2c(C)cccc2Cl)cc1C#Cc1cnc2ccccc2c1]

N′-(2-chloro-6-methylbenzoyl)-4-methyl-3-[2-(3-quinolyl)ethynyl]-benzohydrazide can be prepared by following the procedure known to a person of ordinary skilled in the art, for example, the procedure disclosed in the ‘416 publication.

Surprisingly, N′-(2-chloro-6-methylbenzoyl)-4-methyl-3-[2-(3-quinolyl)ethynyl]-benzohydrazide was found to be a potent c-Abl inhibitor when tested for its ability to inhibit cell proliferation of K562 cells which is a measure of ability of a compound to inhibit endogenous Bcr-Abl kinase activity. The potency of Compound 1 is significantly higher which is surprising and unexpected because the similar compounds from the ‘416 publication failed to show potency at a dose of 0.1 nM as shown in below. (For experimental procedure, refer to the ‘416 publication under In-vitro Cell Proliferation Assay heading).

TABLE 1

In-vitro Cell Proliferation Assay data

| Compound No. | Structure | % inhibition (at 0.1 nM) |
|---|---|---|
| Compound 1 | 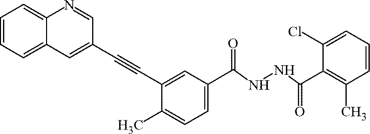 [SMILES: Cc1ccc(C(=O)NNC(=O)c2c(C)cccc2Cl)cc1C#Cc1cnc2ccccc2c1] | 37 |
| Compound 1.10 of the ‘416 publication | 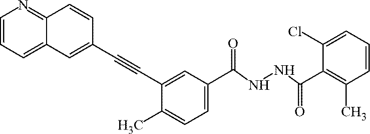 [SMILES: Cc1ccc(C(=O)NNC(=O)c2c(C)cccc2Cl)cc1C#Cc1ccc2ncccc2c1] | 0 |
| Compound 1.21 of the ‘416 publication | 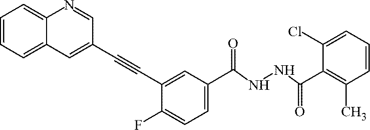 [SMILES: Cc1cccc(Cl)c1C(=O)NNC(=O)c1ccc(F)c(C#Cc2cnc3ccccc3c2)c1] | 6 |
| Compound 1.50 of the ‘416 publication | 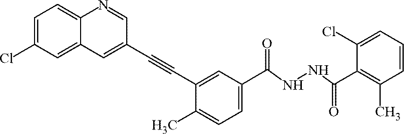 [SMILES: Cc1ccc(C(=O)NNC(=O)c2c(C)cccc2Cl)cc1C#Cc1cnc2ccc(Cl)cc2c1] | 0 |

| | | |
|---|---|---|
| Compound 1.55 of the ‘416 publication | 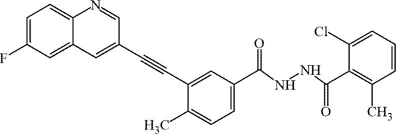 [SMILES: Cc1ccc(C(=O)NNC(=O)c2c(C)cccc2Cl)cc1C#Cc1cnc2ccc(F)cc2c1] | 0 |
| Compound 1.59 of the ‘416 publication | 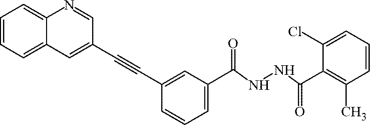 [SMILES: Cc1cccc(Cl)c1C(=O)NNC(=O)c1cccc(C#Cc2cnc3ccccc3c2)c1] | 2 |

Compound 1 has good brain penetration with a ratio of concentration in brain to plasma found to be significantly higher for N′-(2-chloro-6-methylbenzoyl)-4-methyl-3-[2-(3-quinolyl)ethynyl]-benzohydrazide as compared to that for nilotinib or dasatinib. (Example 2 of WO2017/208267, which is incorporated herein by reference in its entirety).

TABLE 2

Concentration of compounds in brain and plasma

| Compound | Treatment (mg/kg) | Time Points (hrs) | Plasma conc. (ng of compound/mL of plasma | Brain conc. (ng of compound/g of brain tissue | Ratio of brain conc./plasma conc. |
|---|---|---|---|---|---|
| Compound 1 | 30 | 1 | 4798±858 | 1901±959 | 0.40 |
| | | 4 | 3167±50 | 510±367 | 0.16 |
| | | 8 | 2715±379 | 435±157 | 0.16 |
| Nilotinib | 100 | 1 | 31683±7958 | 380±70 | 0.01 |
| | | 4 | 38813±11635 | 487±126 | 0.01 |
| | | 8 | 16988±2133 | 180±46 | 0.01 |

As evidenced from the results, Compound 1 showed about 40 times (at 1 hour) higher ratio of brain to plasma concentration compared to nilotinib. Similar results were observed for Compound 1 when compared with dasatinib wherein Compound 1 showed significantly better brain penetration than dasatinib. The results indicate that the Compound 1 shows higher brain concentration for longer duration of time compared with nilotinib and dasatinib.

Accordingly, certain embodiments of the present invention involve uses or methods of administering Compound 1 in such a manner so as to achieve a ratio of brain/plasma concentration of from about 0.16 to about 0.40. Other embodiments involve uses or methods of administering Compound 1 in such a manner so as to achieve a ratio of brain/plasma concentration of 0.16 to 0.40. In these embodiments, the brain/plasma concentrations are measured as they are measured in the Table above.

Compound 1 is also a safe c-Abl inhibitor. Other c-Abl inhibitors are known for cardiovascular toxicity (See J Clin Oncol 33:4210-4218, 2015 and Vascular Health and Risk Management 13: 293-303, 2017). In fact the approved US FDA Label for Tasigna® (nilotinib), Sprycel® (dasatinib), Iclusig® (ponatinib), Bosulif® (bosutinib) and Gleevec® (imatinib) cautions about the sever cardio-toxicity associated with them. Surprisingly, the inventors have found that Compound 1, at a therapeutically effective dose, is devoid of cardiovascular side effects when tested for its in vitro effect on hERG channel and its in vivo effect on ECG parameters like QT interval, QTc interval, QT_{Cf} interval and heart rate in conscious beagle dogs and guinea pig. (Examples 5 and 6 of WO2017/208267).

Thus, according to a first aspect of the present invention, there is provided a method for preventing or treating Alzheimer’s disease and symptoms thereof comprising administering to a subject in need thereof a therapeutically effective amount of N′-(2-chloro-6-methylbenzoyl)-4-methyl-3-[2-(3-quinolyl)ethynyl]-benzohydrazide or its pharmaceutically acceptable salts.

As used herein, “therapeutically effective amount” is a sufficient amount of Compound 1 or a pharmaceutically acceptable salt thereof to provide a therapeutic benefit for the treatment or management of the disease or to delay, minimize or eradicate symptoms associated with the disease.

As used herein the term “pharmaceutically acceptable”, refers to a compound that is not biologically or otherwise undesirable, i.e., the Compound 1 may be incorporated into a pharmaceutical formulation of the invention and administered to a patient without causing any undesirable biological effects or interacting in a deleterious manner with any of the other components of the formulation in which it is contained.

As used herein the term “salts”, refers to salts of an inorganic acid such as hydrochloric acid, hydrobromic acid, phosphoric acid, and the like or salts of organic acids such as, for example, acetic acid, benzenesulfonic acid, methanesulfonic acid, benzoic acid, citric acid, glycolic acid, lactic acid, fumaric acid, succinic acid, adipic acid, pimelic acid, suberic acid, azelaic acid, malic acid, tartartic acid, or amino acids, such as glutamic acid or aspartic acid, and the like.

As used herein the term “subject” refers to a patient such as human suffering from Alzheimer’s disease and who needs therapeutic intervention for the treatment and prevention of AD and symptoms thereof. The patient includes the elderly, for example, those 60 years of age and above.

Studies in an animal model of AD have shown that an increase in Aβ causes increase in c-Abl activity in hippocampal neurons (Schlatterer SD et al, J Mol Neurosci. 2011 Nov; 45(3): 445-452). Inhibition and modulation of c-Abl tyrosine kinase has been shown to decrease tau phosphorylation and behavioral symptoms. Aβ sequence results in two major Aβ isoforms: Aβ42 (42 residue long) and Aβ40 (40 residue long). Aβ42 is the major component of amyloid plaques in AD brains, while Aβ40 is detected only in a subset of plaques. Thus, Aβ42 is the major, and sometimes only, component in amyloid plaques. (See J. Neurochem. 2013; 126, 305-311). Increased Aβ42/Aβ40 ratios appear to correlate with the early-onset familial AD cases caused by presenilin mutations (See Human Mutation 2006, 27(7): 686-695). Lowering Aβ42/Aβ40 ratios in transgenic mice decreases Aβ deposition (See, The Journal of Neuroscience, 2007, 27(3):627-633). Higher neurotoxicity has been reported with samples of higher Aβ42/Aβ40 ratios (See, The EMBO Journal, 2010 29:3408-3420).

Surprisingly, Compound 1showed a dose-dependent reduction in Aβ42 content and the Aβ42/40 ratio. Preferably, the compound disclosed herein decreases the spread of Aβ42 amyloid plaques.

In one embodiment of the present invention, N′-(2-chloro-6-methylbenzoyl)-4-methyl-3-[2-(3-quinolyl)ethynyl]-benzohydrazide or its pharmaceutically acceptable salt regulate levels of tau hyperphosphorylation in AD patients via the inhibition of c-Abl-kinase.

In another embodiment of the present invention, N′-(2-chloro-6-methylbenzoyl)-4-methyl-3-[2-(3-quinolyl)ethynyl]-benzohydrazide or its pharmaceutically acceptable salt prevents or reverses aggregation of Aβ (decreases the spread of Aβ amyloid plaques) in AD patients via the inhibition of Abl-kinase

In yet another embodiment of the present invention, N′-(2-chloro-6-methylbenzoyl)-4-methyl-3-[2-(3-quinolyl)ethynyl]-benzohydrazide or its pharmaceutically acceptable salt regulates levels of tau hyperphosphorylation and at the same time prevents or reverses the aggregation of Aβ (decreases the spread of Aβ plaques) in AD patients via the inhibition of c-Abl-kinase.

In yet another embodiment, N′-(2-chloro-6-methylbenzoyl)-4-methyl-3-[2-(3-quinolyl)ethynyl]-benzohydrazide or its pharmaceutically acceptable salt decreases the spread of Aβ42 amyloid plaques in AD patients via the inhibition of Abl-kinase.

In yet another embodiment of the present invention, there is provided a method for treating and/or preventing AD and symptoms thereof, wherein N′-(2-chloro-6-methylbenzoyl)-4-methyl-3-[2-(3-quinolyl)ethynyl]-benzohydrazide or its pharmaceutically acceptable salt is administered at a dose in the range of about 1.0 mg/kg to about 10.0 mg/kg.

In certain embodiments, the Compound 1 may be administered up to a dose of about 10 mg/kg, preferably from about 1.0 mg/kg to about 10.0 mg/kg, more preferably from about 3.0 mg/kg to about 7.0 mg/kg. In various embodiments, the dose is preferably given once per day, but may also be given in multiple doses per day, for example, once, twice, three times, or four times a day. Alternatively, the dose may be given every other day or every three days, four days, or five days, as will be appreciated by the skilled practitioner. Doses of Compound 1 may be administered for a short time period, e.g., weeks, months, or for a longer time period, such as chronic administration, e.g., over several months or years.

According to another aspect of the present invention, there is provided a method for inhibiting tau hyperphosphorylation comprising administering to a subject an amount of compound sufficient to inhibit tau hyperphosphorylation, wherein said compound modulates an ATP-dependent enzyme, wherein said compound is N′-(2-chloro-6-methylbenzoyl)-4-methyl-3-[2-(3-quinolyl)ethynyl]-benzohydrazide or its pharmaceutically acceptable salt.

Yet another aspect of the present invention provides a method for inhibiting, preventing or reversing aggregation of Aβ plaque comprising administering to an amount of compound sufficient to reduce Aβ plaque, wherein said compound modulates an ATP-dependent enzyme, wherein said compound is N′-(2-chloro-6-methylbenzoyl)-4-methyl-3-[2-(3-quinolyl)ethynyl]-benzohydrazide or its pharmaceutically acceptable salt.

In one embodiment, the enzyme is a kinase.

In another embodiment, the kinase is a tyrosine kinase.

In another embodiment, the tyrosine kinase is c-Abl kinase.

In accordance with another embodiment, N′-(2-chloro-6-methylbenzoyl)-4-methyl-3-[2-(3-quinolyl)ethynyl]-benzohydrazide or its pharmaceutically acceptable salt is further administered in combination with an additional therapeutic agent selected from memantine, donepezil (Aricept®), galantamine (Reminyl®), tacrine hydrochloride (Cognex®), or rivastigmine tartrate (Exelon®).

In another aspect, the present invention provides N′-(2-chloro-6-methylbenzoyl)-4-methyl-3-[2-(3-quinolyl)ethynyl]-benzohydrazide or its pharmaceutically acceptable salt for use in the prevention and/or treatment of Alzheimer’s disease and symptoms thereof.

In yet another aspect, the present invention provides a pharmaceutical composition comprising a therapeutically effective amount of N′-(2-chloro-6-methylbenzoyl)-4-methyl-3-[2-(3-quinolyl)ethynyl]-benzohydrazide or its pharmaceutically acceptable salt for use in the prevention and/or treatment of Alzheimer’s disease and symptoms thereof.

In yet another aspect, the present invention provides the use of N′-(2-chloro-6-methylbenzoyl)-4-methyl-3-[2-(3-quinolyl)ethynyl]-benzohydrazide or its pharmaceutically acceptable salt for the manufacture of a medicament for the prevention and/or treatment of Alzheimer’s disease and symptoms thereof.

According to one embodiment, the phrase “preparation of a medicament” refers to the use of the dosing regimen directly as the medicament in addition to their use in any stage of the preparation of such a medicament.

According to another aspect, there is provided a c-Ab1 inhibitor for use in the prevention and/or treatment of Alzheimer’s disease, risk of developing AD and symptoms thereof, wherein the inhibitor is N′-(2-chloro-6-methylbenzoyl)-4-methyl-3-[2-(3-quinolyl)ethynyl]-benzohydrazide or its pharmaceutically acceptable salt.

According to yet another aspect, there is provided a tau hyperphosphorylation inhibitor for use in the prevention and/or treatment of Alzheimer’s disease and symptoms thereof, wherein the inhibitor is N′-(2-chloro-6-methylbenzoyl)-4-methyl-3-[2-(3-quinolyl)ethynyl]-benzohydrazide or its pharmaceutically acceptable salt.

According to yet another aspect, there is provided an Aβ plaque aggregation inhibitor for use in the prevention and/or treatment of Alzheimer’s disease and symptoms thereof, wherein the inhibitor is N′-(2-chloro-6-methylbenzoyl)-4-methyl-3-[2-(3-quinolyl)ethynyl]-benzohydrazide or its pharmaceutically acceptable salt thereof.

As used herein the term “symptoms” refers to difficulty remembering recent events or conversation, disorientation, mood and behavior, difficulty speaking, swallowing, walking or cognitive disorder that affect learning, memory, perception, problem solving, and include amnesia, dementia and delirium.

Thus, according to another aspect, the present invention provides a method of improving symptoms of Alzheimer’s disease in a subject, comprising administering to the subject a therapeutically effective dose of N′-(2-chloro-6-methylbenzoyl)-4-methyl-3-[2-(3-quinolyl)ethynyl]-benzohydrazide or its pharmaceutically acceptable salt to effectively improve the symptoms in the subject, wherein the improvement is achieved by the inhibition of Aβ plaque aggregation, tau hyperphosphorylation, c-Abl kinase or a combination thereof

In a preferred embodiment, the effective dose of N′-(2-chloro-6-methylbenzoyl)-4-methyl-3-[2-(3-quinolyl)ethynyl]-benzohydrazide or its pharmaceutically acceptable salt ranges from about 1.0 mg/kg to about 10 mg/kg.

In a preferred embodiment, the symptoms of AD include, but are not limited to, memory loss, difficulty performing familiar tasks, problems with language, disorientation to time and place, poor or decreased judgment, problems with abstract thinking, misplacing things, change in mood or behavior, changes in personality and loss of initiative. In another preferred embodiment, the symptoms include cognitive disorder that affect learning, memory, perception, problem solving, and include amnesia, dementia and delirium.

In a preferred embodiment, the pharmaceutical composition is meant for oral administration. The composition suitable for oral use includes, but not limited, to tablets, pellets, capsules, dispersible tablets, sachets, granules, syrup, and the like. The pharmaceutical composition of the present invention can be obtained by conventional approaches using conventional pharmaceutically acceptable excipients well known in the art. Remington’s Pharmaceutical Sciences, Sixteenth Edition, E. W. Martin (Mack Publishing Co., Easton, Pa., 1980) discloses pharmaceutically acceptable excipients which can be used for preparation of a suitable dosage form containing N′-(2-chloro-6-methylbenzoyl)-4-methyl-3-[2-(3-quinolyl)ethynyl]-benzohydrazide or a pharmaceutically acceptable salts thereof as an active ingredient.

For example, pharmaceutically acceptable excipients suitable for tablets preparation include, but not limited to, diluents (e.g., calcium phosphate- dibasic, calcium carbonate, lactose, glucose, microcrystalline cellulose, cellulose powdered, silicified microcrystalline cellulose, calcium silicate, starch, starch pregelatinized, or polyols such as mannitol, sorbitol, xylitol, maltitol, and sucrose), binders (e.g., starch, pregelatinized starch, carboxymethyl cellulose, sodium cellulose, microcrystalline cellulose, hydroxyproyl cellulose, hydroxypropyl methylcellulose, polyvinylpyrrolidone, crospovidone, or combinations thereof), disintegrants (e.g., crosslinked cellulose, crosslinked-polyvinylpyrrolidone (crosspovidone), sodium starch glycolate, polyvinylpyrrolidone (polyvidone, povidone), sodium carboxymethylcellulose, crosslinked sodium carboxymethylcellulose (croscarmellose sodium), hydroxypropyl cellulose, hydroxypropyl methylcellulose, xanthan gum, alginic acid, or soy polysaccharides), wetting agents (e.g., polysorbate, sodium lauryl sulphate, or glyceryl stearate) or lubricants (e.g., sodium lauryl sulfate, talc, magnesium stearate, sodium stearyl fumarate, stearic acid, glyceryl behenate, hydrogenated vegetable oil, or zinc stearate).

In another preferred embodiment, the pharmaceutical composition has a form selected from tablets, pellets, capsules, dispersible tablets, sachets, granules or syrups.

In yet another preferred embodiment, the pharmaceutical composition is a capsule and is administered orally.

In yet another preferred embodiment, the pharmaceutical composition is a tablet and is administered orally.

### Biological Evaluation

Hereinafter, the present invention is described with reference to test examples, but the scope of the preset invention is not limited thereto. Any modification in the procedures described herein, other assays or models and modification thereon can be employed or adapted. All such modifications and alternative procedures are within the spirit and scope of the present application.

### Evaluation of Compound 1 in a Mouse Model of Alzheimer’s Disease

Efficacy of Compound 1 was evaluated in two transgenic mouse models- Double transgenic APP/PS1 and Triple transgenic Tg-SwDI mouse models of AD. APP/PS1 are double transgenic mice expressing a chimeric mouse/human amyloid precursor protein (Mo/HuAPP695swe) and a mutant human presenilin 1 (PS1-dE9), both directed to CNS neurons. Both mutations are associated with early-onset Alzheimer’s disease. The “humanized” Mo/HuAPP695swe transgene allows the mice to secrete a human Aβ peptide. These mice may be useful in studying neurological disorders of the brain, specifically Alzheimer’s disease, amyloid plaque formation and aging. Triple transgenic Tg-SwDI mice express neuronally derived human amyloid beta-precursor protein, APP gene, 770 isoform, containing the Swedish mutations (Lys670→Asn/ Met671→Leu), Dutch (Glu693→Gln) and Iowa (Asp694→Asn) mutations, under the control of the mouse thymus cell antigen 1, theta, Thy1, promoter.

### Study I

Six month old male APP/PS1 mice which show little overt behavioral or pathological phenotype of AD were used. All experiments involving animals were performed in accordance with institutional guidelines for the use and care of animals after approval from the Institutional Animal Ethics Committee (IAEC), Central Animal Facility, Indian Institute of Science. Only male mice were used for the study and were housed at ambient temperature of 25° C. under 12 hours light-dark cycle with free access to food chow and drinking water. All mice were dosed once a day, orally with placebo for Compound 1 at 30 and 45 mg/kg for a period of three months.

After completion of treatment phase, mice were subjected to behavioural assessment using CFC test, followed by biochemical estimation of brain Aβ using an enzyme-linked immunosorbent assay (ELISA) and Aβ plaque load using Immunohistochemistry (IHC).

### Total Aβ Content in Cortex of Tg APP/PS1 Mice Using ELISA

The animals were (n=10/treatment) deeply anesthetized with a mixture of xylazine and ketamine (1:8), perfused with 10 mL of 1x saline for 4 minutes, their brains were excised, homogenized immediately in 0.5 mL ELISA buffer, and centrifuged at 5000xg for 15 minutes at 4° C. to precipitate the pellet. Total Aβ was estimated in the cortex of APP/PS1 mice using ELISA assay kit as per the manufacturer instruction.

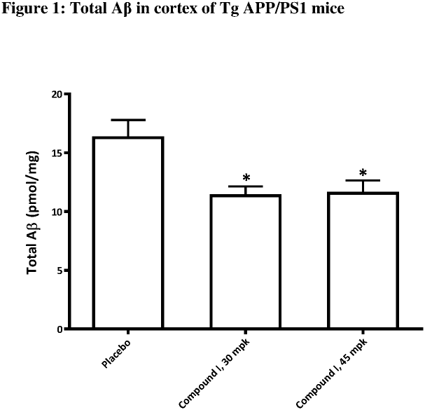

Compound 1 at 30 and 45 mg/kg dose levels showed significant reduction in total Aβ content in cortex of Tg APP/PS1 mice compared to placebo treated group. (See FIG. 1)

### Aβ Plaque Load (% Area Fraction) in Cortex of Tg APP/PS1 Mice Using IHC

Mouse brains (n=5/treatment) were fixed in 4% (w/v) buffered paraformaldehyde (PFA). The brain tissues were processed for paraffin embedding and serial sections (5 µM thickness) were cut using microtome (Leica Biosystems Inc., Buffalo Grove, IL, USA). Sections were de-waxed by xylene treatment followed by a brief wash with chloroform to remove the residual xylene. Endogenous peroxidase activity was blocked with methanol containing hydrogen peroxide (3%, v/v) for 30 minutes. Sections were hydrated in graded alcohol followed by brief washings with water and phosphate buffered saline (10 mM, pH 7.4, PBS). The sections were pressure cooked for 10 minutes in sodium citrate buffer (pH 6.8). After cooling, the sections were washed with Phosphate-buffered saline (PBS) and incubated in normal goat serum for 30 minutes at room temperature to block non-specific binding. Next, the sections were incubated with primary antibody anti-β-Amyloid, 1-16 antibody (6E10) overnight at 4° C. After washing with phosphate buffered saline (10 mM, pH 7.4, PBS), the sections were incubated in secondary antibody. Subsequently, the sections were washed and mounted with anti-fade mounting medium (VECTASHIELD). Image acquisition was performed using 40×/0.75 NA, Zeiss Axio Imager M2 (Carl Zeiss Microscopy, LLC, Thornwood, NY, USA).

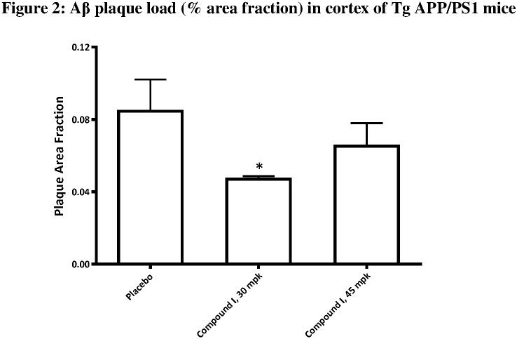

The IHC result showed that compared to placebo, Compound 1 significantly reduced Aβ plaque (% area fraction) at the dose of 30 mg/kg. (See FIG. 2).

### Contextual Fear Conditioning (CFC): Freezing Behaviour in Tg APP/PS1 Mice

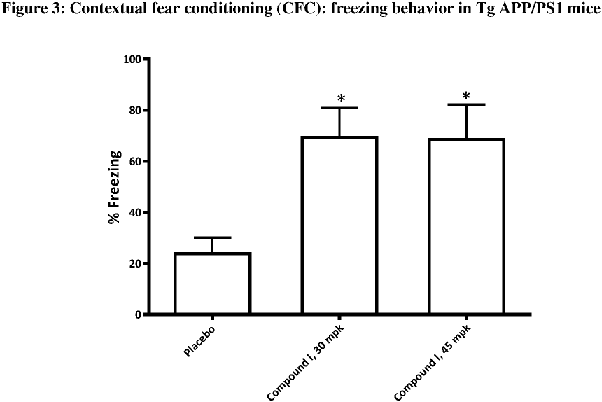

The CFC test was performed using an apparatus from San Diego Instruments (CA, USA). The CFC training context was rectangular in shape, identity of the context was maintained with the presence of distinct odor (2% acetic acid, v/v). The conditioning chamber was cleaned with 70% ethanol before and after each session. Mice were housed singly and were first handled for 5 minutes for 3 days. On training day, mice (n=6/treatment) were allowed to explore the training context for 1 minute and then received 3 foot-shocks (2 s and 0.6 mA each, inter-trial interval 30 s). Contextual fear memory was assessed by returning mice to the training context 24 hours after fear conditioning and analyzing freezing during a test period of 2 minutes. Freezing was defined as complete absence of somatic mobility other than respiratory movements. (Sci Rep.2018; Sep 3; 8(1):13119).The result of CFC analysis showed significant increase in % freezing of mice at 30 and 45 mg/kg doses of Compound 1 as compared to placebo treated group..These results suggest that the treatment with Compound 1 at indicated doses results in improvement in cognitive deficits in Tg APP/PS1 mice. (See FIG. 3).

### Study II

### Estimation of Aβ₄₂ and Aβ₄₂/Aβ₄₀ Ratio in TgSwDI Mice Using ELISA

Female TgSwDI (expresses human APP gene containing Swedish, Dutch, and Iowa mutations) of 5-7 months of age were used for the study. Animals were habituated for handling for one week prior to initiation of dosing. All experiments involving animals were performed in accordance with institutional guidelines for the use and care of animals after approval from the Institutional Animal Ethics Committee (IAEC), Sun Pharma Advanced Research Company (SPARC) Ltd. Mice were housed at an ambient temperature of 25° C. under 12 hours light-dark cycle with free access to food chow and drinking water. Animals were treated with assigned treatments p.o., o.d., for a period of 96 days. Thus, Compound 1 was given at 15, 30, and 45 mg/kg dose and nilotinib was given at 30 mg/kg dose. After completion of treatment phase, mice from each treatment group were perfused with ice-cold phosphate buffered saline (PBS, pH 7.4), brain was excised, cortex was isolated and processed for preparation of lysates as per protocol provided by Invitrogen ELISA kit. Estimation of Aβ42 and Aβ40 were carried out as per the instructions provided by Invitrogen user manual. Ratio of Aβ42/40 was calculated.

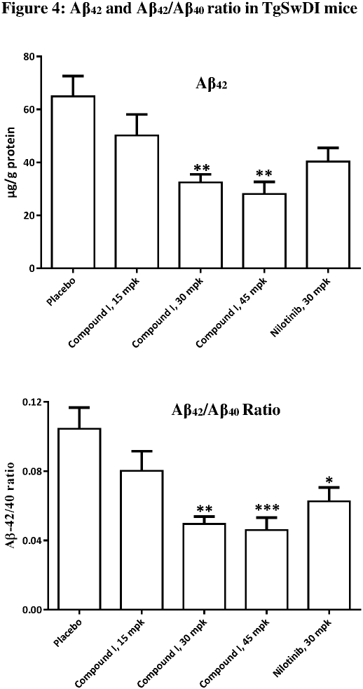

The result of ELISA indicated that Compound 1 at 15, 30, and 45 mg/kg showed dose-dependent reduction in Aβ42 content and the Aβ42/40 ratio. Compared to placebo group, the effect on Aβ42 content and the ratio was statistically significant at 30 and 45 mg/kg doses. Compared to placebo, nilotinib had no statistically significant reduction in Aβ42 content. (See FIG. 4).

We claim:

1. A method for preventing or treating Alzheimer’s disease and symptoms thereof comprising administering to a subject in need thereof a therapeutically effective amount of N′-(2-chloro-6-methylbenzoyl)-4-methyl-3-[2-(3-quinolyl)ethynyl]-benzohydrazide or its pharmaceutically acceptable salts.

2. The method according to claim 1, wherein the N′-(2-chloro-6-methylbenzoyl)-4-methyl-3-[2-(3-quinolyl)ethynyl]-benzohydrazide or its pharmaceutically acceptable salts regulate levels of tau hyperphosphorylation.

3. The method according to claim 1, wherein the N′-(2-chloro-6-methylbenzoyl)-4-methyl-3-[2-(3-quinolyl)ethynyl]-benzohydrazide or its pharmaceutically acceptable salts regulate levels of tau hyperphosphorylation by inhibition of c-Abl-kinase.

4. The method according to claim 1, wherein the N′-(2-chloro-6-methylbenzoyl)-4-methyl-3-[2-(3-quinolyl)ethynyl]-benzohydrazide or its pharmaceutically acceptable salts prevent or reverse aggregation of Aβ.

5. The method according to claim 1, wherein the N′-(2-chloro-6-methylbenzoyl)-4-methyl-3-[2-(3-quinolyl)ethynyl]-benzohydrazide or its pharmaceutically acceptable salts prevent or reverse aggregation of Aβ by inhibition of c-Abl-kinase.

6. The method according to claim 1, wherein the N′-(2-chloro-6-methylbenzoyl)-4-methyl-3-[2-(3-quinolyl)ethynyl]-benzohydrazide or its pharmaceutically acceptable salt is administered at a dose in a range of about 1.0 mg/kg to about 10.0 mg/kg.

7. A method for inhibiting tau hyperphosphorylation comprising administering to a subject an amount of compound sufficient to inhibit tau hyperphosphorylation, wherein said compound modulates an ATP-dependent enzyme, wherein said compound is N′-(2-chloro-6-methylbenzoyl)-4-methyl-3-[2-(3-quinolyl)ethynyl]-benzohydrazide or its pharmaceutically acceptable salt.

8. A method for inhibiting, preventing or reversing aggregation of Aβ comprising administering to a subject an amount of compound sufficient to reduce Aβ plaque, wherein said compound modulates an ATP-dependent enzyme, wherein said compound is N′-(2-chloro-6-methylbenzoyl)-4-methyl-3-[2-(3-quinolyl)ethynyl]-benzohydrazide or its pharmaceutically acceptable salt.

9. The method according claim 7, wherein the ATP-dependent enzyme is a kinase.

10. The method according to claim 9, wherein the kinase is a tyrosine kinase.

11. The method according to claim 10, wherein the tyrosine kinase is c-Abl kinase.

12. The method according to claim 1, wherein the N′-(2-chloro-6-methylbenzoyl)-4-methyl-3-[2-(3-quinolyl)ethynyl]-benzohydrazide or its pharmaceutically acceptable salts is further administered in combination with an additional therapeutic agent.

13. The method according to claim 12, wherein the additional therapeutic agent according is selected from memantine, donepezil, galantamine, tacrine hydrochloride, or rivastigmine tartrate.

14. (canceled)

15. A pharmaceutical composition comprising a therapeutically effective amount of N′-(2-chloro-6-methylbenzoyl)-4-methyl-3-[2-(3-quinolyl)ethynyl]-benzohydrazide or its pharmaceutically acceptable salts for use in the prevention and/or treatment of Alzheimer’s disease and symptoms thereof.

16. The pharmaceutical composition according to claim 15, wherein the pharmaceutical composition has a form selected from tablets, pellets, capsules, dispersible tablets, sachets, granules or syrups.

17. The pharmaceutical composition according to claim 16, wherein the pharmaceutical composition is a capsule confiqured to be administered orally.

18. The pharmaceutical composition according to claim 16, wherein the pharmaceutical composition is a tablet cconfigured to be administered orally.

19. A method of making a medicament for the prevention and/or treatment of Alzheimer’s disease and symptoms thereof comprising combining N′-(2-chloro-6-methylbenzoyl)-4-methyl-3-[2-(3-quinolyl)ethynyl]-benzohydrazide or its pharmaceutically acceptable salts with a pharmaceutically acceptable excipient.

20. A method of inhibiting c-Abl comprising administering to a subject N′-(2-chloro-6-methylbenzoyl)-4-methyl-3-[2-(3-quinolyl)ethynyl]-benzohydrazide or its pharmaceutically acceptable salts in an amount sufficient to inhibit c-Abl.

21. A method of inhibiting tau hyperphosphorylation comprising administering to a subject N′-(2-chloro-6-methylbenzoyl)-4-methyl-3-[2-(3-quinolyl)ethynyl]-benzohydrazide or its pharmaceutically acceptable salts in an amount sufficient to inhibit tau hyperphosphorylation.

22. A method of inhibiting Aβ plaque aggregation comprising administering to a subject N′-(2-chloro-6-methylbenzoyl)-4-methyl-3-[2-(3-quinolyl)ethynyl]-benzohydrazide or its pharmaceutically acceptable salts in an amount sufficient to inhibit Aβ plaque aggregation.

23. A method of improving symptoms of Alzheimer’s disease in a subject comprising administering to the subject a therapeutically effective dose of N′-(2-chloro-6-methylbenzoyl)-4-methyl-3-[2-(3-quinolyl)ethynyl]-benzohydrazide or its pharmaceutically acceptable salts, thereby improving the symptoms in the subject, wherein improvement is achieved by inhibition of Aβ plaque aggregation, tau hyperphosphorylation, c-Abl kinase or combination thereof.

24. The method according to claim 23, wherein the N′-(2-chloro-6-methylbenzoyl)-4-methyl-3-[2-(3-quinolyl)ethynyl]-benzohydrazidesempe, or its pharmaceutically acceptable salts is administered at a dosage in a range of from about 1.0 mg/kg to about 10.0 mg/kg.

25. The method according to claim 23 in which the symptoms of Alzheimer’s disease are difficulty remembering recent events or conversation, disorientation, mood and behavior, difficulty speaking, swallowing, walking or cognitive disorder.

26. The method or use according to claim 23, wherein the improvement is achieved by inhibition of c-Abl kinase, and wherein a percentage inhibition of c-Abl kinase is 37%.

27. The method or use according to claim 23, wherein the improvement is achieved by inhibition of c-Abl kinase, and wherein a percentage inhibition of c-Abl kinase is about 37%.

28. The method or use according to claim 23, wherein the improvement is achieved by inhibition of c-Abl kinase, and wherein the percentage inhibition of c-Abl kinase is at least 37%.
